(19) United States
(12) Patent Application Publication
(10) Pub. No.: US 2021/0076940 A1
(43) Pub. Date: Mar. 18, 2021

(54) DEVICE FOR REGISTERING AND FOR DESTROYING INDIVIDUAL TUMOR CELLS, TUMOR CELL CLUSTERS, AND MICROMETASTASES IN THE BLOODSTREAM

(71) Applicants: Detlef SCHIKORA, Beverungen (DE); Michael WEBER, Lauenfoerde (DE)

(72) Inventors: Detlef SCHIKORA, Beverungen (DE); Michael WEBER, Lauenfoerde (DE)

(21) Appl. No.: 16/772,338

(22) PCT Filed: Dec. 13, 2018
(86) PCT No.: PCT/DE2018/101015

§ 371 Date: Jun. 12, 2020

(30) Foreign Application Priority Data
Dec. 14, 2017 (DE) 10 2017 129 971.8

(51) Int. Cl. A61B 5/00; A61B 5/1459; A61N 5/06

(52) U.S. Cl. CPC A61B 5/0075; A61B 5/0086; A61B 5/4836; A61B 5/1459; A61N 2005/0626; A61B 5/6876; A61N 5/062; A61N 2005/0602; A61B 5/6852

(57) ABSTRACT

The present invention relates to a device for detecting individual tumor cells, tumor clusters and micro metastases in the circulatory system which are enriched with a photo-sensitive substance. It is the object of the invention to provide a device allowing both a secure diagnostic detection of individual tumor cells in the circulatory system of humans and mammals and their therapeutic elimination in situ. This task is solved by a device comprising a radiation source (7, 11) with intravascular or extravascular excitation of the photo-sensitive substance, a detector (1, 12, 13) with intravascular or extravascular detection of a fluorescence and/or phosphorescence and/or luminescence radiation of the excited tumor cells and/or tumor clusters and/or micro metastases, a high sensitivity fluorescence spectrometer (5), connected to said detector (1, 12, 13), for detecting the emitted radiation, a computer (6) connected to said spectrometer (5) recording the received peaks of the emitted radiation as a function of time.

### CROSS REFERENCE TO RELATED APPLICATIONS

This application is the National Stage of PCT/DE2018/101015 filed on Dec. 13, 2018, which claims priority under 35 U.S.C. § 119 of German Application No. 10 2017 129 971.8 filed on Dec. 14, 2017, the disclosure of which is incorporated by reference. The international application under PCT article 21(2) was not published in English.

The invention relates to a device for detecting and for destroying individual tumor cells, tumor clusters and micro metastases in the circulatory system, which are enriched with a photo-sensitive substance.

Using the biophysical methods available today in routine oncology diagnostics, such as magnetic resonance (MRT; fMRI), positron emission tomography (PET), ultrasound (US), and computer aided tomography (CT) and X-ray, tumors can be detected directly provided they have an average tumor mass of 1 g. This corresponds to about 1 billion (10⁹) tumor cells. Also, it is only from this size on upwards that tissue samples can be sampled directly (biopsy) and used for clinically proven diagnostics.

Thus, a tumor mass of 1 g constitutes the presently valid diagnostic threshold. The findings determined at this diagnostic threshold are considered by oncologists as “early detection” of tumor growth or, respectively, metastasis.

By means of immunological methods tumors can be detected that have a tumor mass of 1 mg and correspondingly consists of about 1 million (10⁶) tumor cells. These methods are indirect in nature because they rely on the determination of marker substances. Tumor markers are substances which are generated directly by malignant tumor cells or whose synthesis in non-tumor cells is induced by tumor cells and which also occur in healthy people.

When tumor markers appear in increased concentrations in the blood or in other body fluids (humoral tumor markers) or, respectively, in or on cells (cellular tumor markers), the enable inferences as to the existence, the course and the prognosis of a tumor disease. These tumor markers are, generally, non-specific. Thus, a high concentrations of the tumor marker CEA in the blood analysis indicates that there could be a lung carcinoma or a liver carcinoma or a colon carcinoma or pancreatic carcinoma or a breast carcinoma or a stomach carcinoma.

Tumor cells or, respectively, micro metastases which, virtually unavoidably, may still be present in the body after an operation may lead to recurrence of the disease at a later time. In the present state of the art, using ultrasound or computer aided or magnetic resonance imaging, it is impossible, either during an operation or thereafter, to detect such small cell clusters (so-called micro metastases). However, experience shows that at this stage, i.e. upon completion of chemotherapy, the risk of recurrence of a tumor and formation of metastases is still relevant and not negligible for virtually all cancer patients. Presently, all that medicine, in particular oncology, has to offer for this phase of the disease is the principle “monitor and be optimistic.” There are no therapeutic options available because of the lack of diagnostic basis here for.

Therefore, it is the object of the present invention to provide a device allowing both a secure diagnostic detection of individual tumor cells in the circulatory system of humans and mammals and the therapeutic in-situ-elimination thereof.

The basis of the present invention lies in photodynamic diagnostics (PDD) as well as in photodynamic therapy (PDT) both of which are successfully employed in cancer therapy internationally and recognized in orthodox medical science.

Both PDD and PDT are based on the optical excitement of photo-sensitive substances which are being applied to the organism topically or systemically by means of infusion and which accumulate in tumor cells because these, independent of the type of tumor, always exhibit a higher rate of cell metabolism than normal, healthy cells.

The optical excitation of such light sensitive substances (photo sensitizer) leads to the generation of reactive oxygen (singulate oxygen) in the tumor cell and to the destruction of essential cell components (mitochondria, cell walls) by oxidation, ending at necrotic or apoptotic cell death. The theory of this process has been described by means of the Jablonski diagram. (See A. Jablonski: Efficiency of anti-Stokes fluorescence in dyes, Nature, Vol. 131, (1933), at 839-840)

The Jablonski diagram states that the process of generating reactive oxidative species is accompanied and characterized by the creation and emission of photons. Thus, upon relaxation of the respective light sensitive molecule from a first excited state (single state) into the ground state fluorescence radiation is generated, while upon transition from a further excited state (triplet state) into the ground state phosphorescence radiation is generated, and upon relaxation from the excited singulate oxygen to molecular oxygen characteristic luminescence radiation is generated.

These three different photon emissions constitute a characteristic of a photo-dynamic process (PDD and PDT) and are the definite physical identification of an activated photo-dynamic event. This event happens in each tumor cell enriched with a photosensitizer and optically activated, and, moreover, of course, also in each micro metastasis and in each macroscopic tumor, independent of the type of tumor.

The intensity of the radiation emitted is directly proportional to the number of photo-dynamically activated tumor cells.

In the state of the art, it is possible to detect individual photons by means of modern optical detectors (see A. Korneev, G. Goltsman, W. H. P. Pernice “Single Photon Counting: Photonic integration meets single photon detection” LaserFocus World V.51ls.5.05/05/2015).

For detecting individual photons high gain detectors are used. These include photomultipliers (PMT) and avalanche photo diodes (APD).

According to the invention, the task presented above is solved by means of a device having the features of claim 1.

This drastically lowers the diagnostic threshold for tumor identification and tumor therapy because it is possible to diagnose and treat individual tumor cells or micro metastases. It is possible to carry out an early tumor detection in the true sense of the word. This applies in general to all patients with an increased genetic risk of cancer in the sense of a prophylactic therapy as well as to all tumor patients following an adjuvant chemotherapy or palliative chemo/radiation therapy and to veterinary medicine.

The invention is further illustrated below by means of embodiment examples. The accompanying drawing shows schematically:

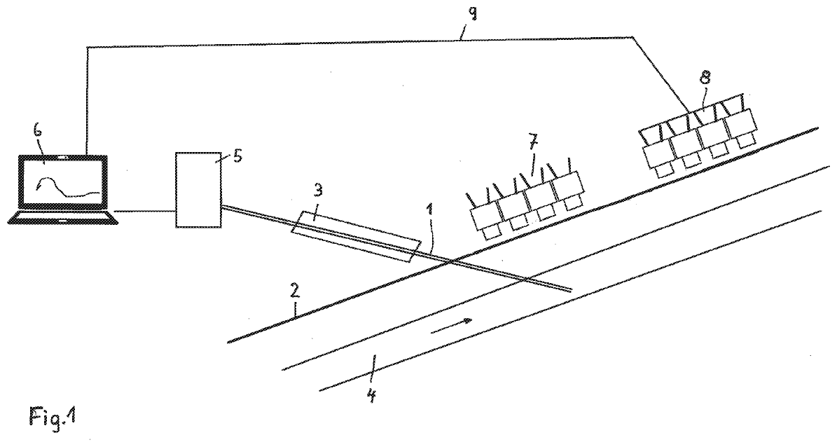

FIG. 1 a first embodiment of a device according to the invention with extravascular excitation of tumor cells and intravascular detection of the radiation emitted,

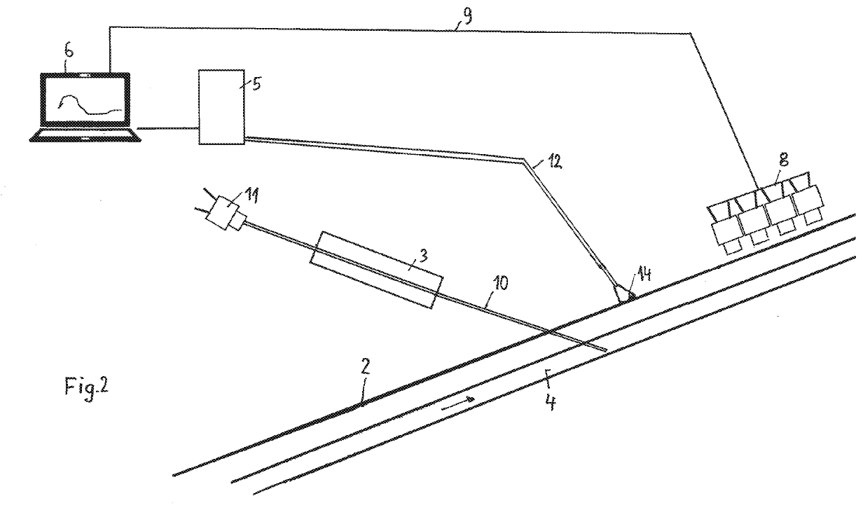

FIG. 2 a second embodiment of a device according to the invention with intravascular excitation of tumor cells and intravascular detection of the radiation emitted,

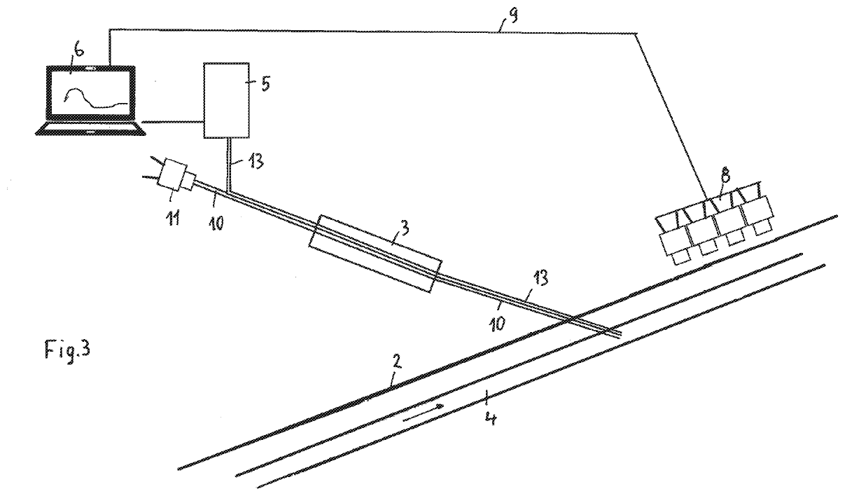

FIG. 3 a third embodiment of a device according to the invention with intravascular excitation of tumor cells and intravascular detection of the radiation emitted, and

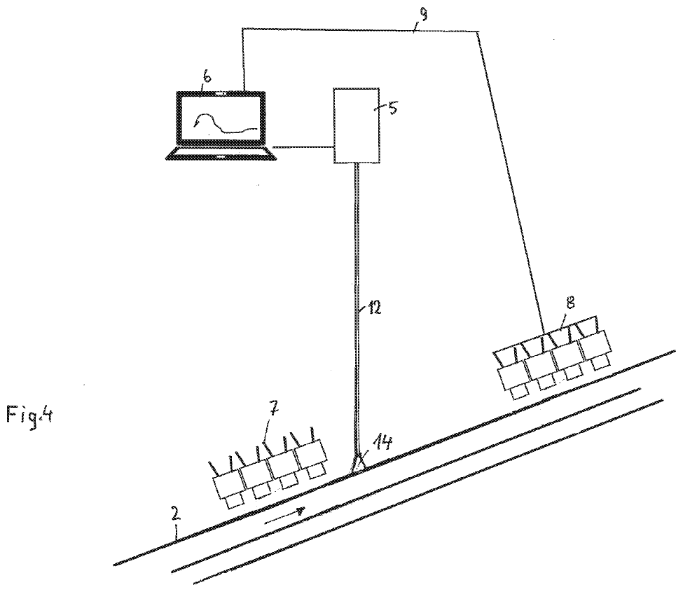

FIG. 4 a fourth embodiment of a device according to the invention with extravascular excitation of tumor cells and extravascular detection of the radiation emitted.

In the embodiment according to FIG. 1, a device according to the invention comprises a fiber optic cable 1 which is positioned with its inlet in a vein 4 close to the surface by means of a suitable peripheral venous catheter 3 (e.g. using a BraunOle) affixed to the skin 2 of a patient. The outlet of the fiber optic cable 1 is connected to a high sensitivity fluorescence spectrometer or Ramann spectrometer 5 adapted in terms of its spectral position and sensitivity. The spectrometer 5 in turn is connected to a computer 6. On the outside of the skin 2 along the vein 4 an LD/LED field 7, for example 6 cm long and 1.5 cm wide, is arranged such that the inlet surface of the fiber optic cable 1 protrudes into the radiation field of the LD/LED field 7 by approximately 10 mm.

The device described is operated as follows:

Indocyanine green (ICG) is used as photo sensitizer. Through a peripheral infusion line a quantity of ICG adapted to the body weight of the patient is infused into the circulatory system. If tumor cells are still present in the circulatory system these will become enriched with the photo sensitizer more strongly compared to normal blood cells because the rate of metabolism is higher.

Following an exposure time of about 3 hours the peripheral venous catheter 3 is placed, for example, into the vena basilica or another superficial vein 4 of the arm or hand. The fiber optic cable 1 is introduced into the venous catheter 3 and placed as described above.

It is a known fact that ICG is optically excited by radiation at a wavelength of 780 nm. The LD/LED field 7 emits infrared radiation at this very wavelength. When this is switched on the ICG evenly distributed inside the bloodstream is activated. The fluorescence, phosphorescence, and luminescence radiation created thereby enters the radiation entry surface of the fiber optic cable 1 and, from there, is guided to the spectrometer 5. In the spectrometer 5 this radiation is analyzed and represented as functional correlation of the radiation intensity over wavelength. The base spectrum generated in this manner is subtracted from itself by means of a suitable software so that a differential spectrum is created and displayed in the connected computer 6 the intensity of which as a function of wavelength is equal to zero.

Now, when a tumor cell or a tumor cluster enriched with ICG flows into the radiation area of the LD/LED field 7, this will emit fluorescence, phosphorescence and luminescence radiation, the intensities of which are greater in any case than zero and, therefore, appear in the spectrum as intensity peaks.

The spectrum is measured continuously over a period of time t1 which is chosen such that the entire blood volume has passed the fiber optic cable 1 and the radiation area of the LD/LED field 7 a plurality of times, preferably two to three times.

If the spectrum registered for the measuring period t1 exhibits no peaks then this is a safe indication that no radiation above the base intensity was detected and, therefore, no circulating tumor cells are present.

In the event, however, that the spectrum registered for the measuring period t1 exhibits peaks at 830 nm (fluorescence radiation) and/or 945 nm (phosphorescence radiation) and/or 1245 nm (luminescence radiation), this will indicate the presence of circulating tumor cells in the bloodstream. This concludes the photo-dynamic diagnostics (PDD) of the bloodstream.

If radiation peaks are being detected in the PDD then the photo-dynamic therapy (PDT) is applied to destroy these tumor cells in that a further LD/LED field 8, for example having a length of 6 cm long and a width of 1.5 cm, is arranged outside along the vein 4 downstream from the LD/LED field 8 in the direction of blood flow the emissions of which are tuned exactly to the wavelength of the fluorescence radiation detected in the PDD, i.e. 830 nm. Upon registration of a radiation peak in the computer 6 the LD/LED field 8 is activated via computer control for a treatment period t1, for which purpose the computer 6 and the LD/LED field 8 are connected to each other via a line 9. The 830 nm radiation which is absorbed only minimally in the skin 2 enters the bloodstream almost at full intensity and destroys the tumor cells circulating there.

Spreading processes result in the radiation field of the LD/LED field 8 having a length of approximately 10 cm. At an average venous flow velocity of approximately 10 cm/s (e.g. vena femoralis superior) this will result in a retention time of a tumor cell in the radiation field of 1 s. At a presumed average venous flow rate of 400-900 ml/min. this will result in a treatment period t1=7-15 min for each passage, i.e. an effective treatment period of 15-30 min.

Immediately thereafter it is possible to test the result of the PDT by carrying out the above-described spectral blood analysis (PDD) again.

In the alternative, of course, it is also possible to remove the diagnosed tumor cells in a later treatment step using methods of treatment established in oncology (chemotherapy, radiation therapy).

In the embodiment according to FIG. 2 a device according to the invention comprises a fiber optic cable 10 which is positioned with its outlet in a vein 4 close to the surface by means of a suitable peripheral venous catheter 3 (e.g. using a Braunüle) affixed to the skin 2 of a patient. An LD or LED 11 is incorporated in the inlet of the fiber optic cable 10 as radiation source. The inlet of a further fiber optic cable 12 is affixed, above the outlet of the intravenous fiber optic cable 10, to the skin 2 of the patient, the inlet being designed conical so as to enlarge the surface for entry of radiation. The outlet of the fiber optic cable 12 is connected to a high sensitivity fluorescence spectrometer or Ramann spectrometer 5 adapted according to the invention in terms of its spectral position and sensitivity. The spectrometer 5 in turn is connected to a computer 6.

The device described is operated as follows:

Chlorine e6 is used as photo sensitizer. Through a peripheral infusion line a quantity of e6 adapted to the body weight of the patient is infused into the circulatory system. If tumor cells are still present in the circulatory system these will become enriched with the photo sensitizer more strongly compared to normal blood cells because the rate of metabolism is higher.

Following an exposure time of about 3 hours the peripheral venous catheter 3 is placed, for example, into the vena basilica or another superficial vein 4 of the arm or hand. The fiber optic cable 10 is introduced into the venous catheter 3 and placed as described above.

It is a known fact that e6 is optically excited by light at a wavelength of 405 nm (purple blue). The LD/LED 11 incorporated in the fiber optic cable 10 emits light of this wavelength at a power output of 50 mW. When this is switched on the e6 evenly distributed inside the bloodstream is activated. The fluorescence, phosphorescence, and luminescence radiation created thereby enters the entry surface of the fiber optic cable 12 and, from there, is guided to the spectrometer 5. In the spectrometer 5 this radiation is analyzed and represented as functional correlation of the radiation intensity over wavelength. The base spectrum generated in this manner is subtracted from itself by means of a suitable software so that a differential spectrum is created and displayed in the connected computer 6 the intensity of which as a function of wavelength is equal to zero.

Now, when a tumor cell or a tumor cluster enriched with Chlorin e6 flows into the intravascular 405 nm radiation area of the fiber optic cable 10, this will emit fluorescence, phosphorescence and luminescence radiation, the intensities of which are greater in any case than zero and, therefore, appear in the spectrum as intensity peaks.

The spectrum is measured continuously over a period of time t1 which is chosen such that the entire blood volume has passed the intravascular 405 nm radiation area a plurality of times (two to three times).

If the spectrum registered for the measuring period t1 exhibits no peaks then this is a safe indication that no radiation above the base intensity was detected and, therefore, no circulating tumor cells are present.

In the event, however, that the spectrum registered for the measuring period t1 exhibits peaks at 660 nm (fluorescence radiation) and/or 840 nm (phosphorescence radiation) and/or 1245 nm (luminescence radiation), this will indicate the presence of circulating tumor cells in the bloodstream. This concludes the photo-dynamic diagnostics (PDD) of the bloodstream.

If radiation peaks are being detected in the PDD then the photo-dynamic therapy (PDT) is applied to destroy these tumor cells in that a further LD/LED field 8, for example having a length of 6 cm long and a width of 1.5 cm, is arranged outside along the vein 4 downstream from the radiation field of the fiber optic cable 10 in the direction of blood flow the emissions of which are tuned exactly to the wavelength of the fluorescence radiation detected in the PDD, i.e. 660 nm. Upon registration of a radiation peak in the computer 6 the LD/LED field 8 is activated via computer control for a treatment period t1, for which purpose the computer 6 and the LD/LED field 8 are connected to each other via a line 9. The 660 nm radiation which is absorbed only minimally in the skin 2 enters the bloodstream almost at full intensity and destroys the tumor cells circulating there.

Spreading processes result in the radiation field of the LD/LED field 8 having a length of approximately 10 cm. At an average venous flow velocity of approximately 10 cm/s (e.g. vena femoralis superior) this will result in a retention time of a tumor cell in the radiation field of 1 s. At a presumed average venous flow rate of 400-900 ml/min. this will result in a treatment period t1=7-15 min for each passage, i.e. an effective treatment period of 15 to 30 min.

Immediately thereafter it is possible to test the result of the PDT by carrying out the above-described spectral blood analysis (PDD) again.

In the alternative, of course, it is also possible to remove the diagnosed tumor cells in a later treatment step using methods of treatment established in oncology (chemotherapy, radiation therapy).

In the embodiment according to FIG. 3 a device according to the invention comprises a first fiber optic cable 10 auf, is positioned with its outlet in a vein 4 close to the surface by means of a suitable peripheral venous catheter 3 (e.g. using a Braunüle) affixed to the skin 2 of a patient. In the inlet of the first fiber optic cable 10 an LD or LED 11 is incorporated as radiation source. By means of the peripheral venous catheter 3 affixed to the skin 2 of the patient a second fiber optic cable 13 is positioned parallel to the first fiber optic cable 10 intravascularly in the vein 4 such that its inlet lies in the light field of the first fiber optic cable 10 and receives the radiation generated by the optical excitation.

The outlet of the fiber optic cable 13 is connected to a high sensitivity fluorescence spectrometer or Ramann spectrometer 5 adapted in terms of its spectral position and sensitivity. The spectrometer 5 in turn is connected to a computer 6.

The device described is operated as follows:

5-alpha levulinic acid (5-ALA) is used as photo sensitizer. Through a peripheral infusion line a quantity of 5-ALA adapted to the body weight of the patient is infused into the circulatory system. If tumor cells are still present in the circulatory system these will become enriched with the photo sensitizer more strongly compared to normal blood cells because the rate of metabolism is higher.

Following an exposure time of about 3 hours the peripheral venous catheter 3 is placed, for example, into the vena basilica or another superficial vein 4 of the arm or hand. The two fiber optic cables 10 and 13 are introduced into the venous catheter 3 and placed as described above.

It is a known fact that 5-ALA is optically excited by light at a wavelength of 405 nm (purple blue). The LD/LED 11 incorporated in the fiber optic cable 10 emits infrared radiation at this wavelength at a power output of 50 mW. When this is switched on the 5-ALA evenly distributed inside the bloodstream is activated. The fluorescence, phosphorescence, and luminescence radiation created thereby enters the entry surface of the fiber optic cable 13 and, from there, is guided to the spectrometer 5. In the spectrometer 5 this radiation is analyzed and represented as functional correlation of the radiation intensity over wavelength. The base spectrum generated in this manner is subtracted from itself by means of a suitable software so that a differential spectrum is created and displayed in the connected computer 6 the intensity of which as a function of wavelength is equal to zero.

Now, when a tumor cell or a tumor cluster enriched with 5-ALA flows into the intravascular 405 nm radiation area, this will emit fluorescence, phosphorescence and luminescence radiation, the intensities of which are greater in any case than zero and, therefore, appear in the spectrum as intensity peaks.

The spectrum is measured continuously over a period of time t1 which is chosen such that the entire blood volume has passed the intravascular 405 nm radiation area a plurality of times (two to three times) (see infra, between 15 min and 30 min).

If the spectrum registered for the measuring period t1 exhibits no peaks then this is a safe indication that no radiation above the base intensity was detected and, therefore, no circulating tumor cells are present.

In the event, however, that the spectrum registered for the measuring period t1 exhibits peaks at 630 nm (fluorescence radiation) and/or 780 nm (phosphorescence radiation) and/or 1245 nm (luminescence radiation), this will indicate the presence of von circulating tumor cells in the bloodstream. This concludes the photo-dynamic diagnostics (PDD) of the bloodstream.

If radiation peaks are being detected in the PDD then the photo-dynamic therapy (PDT) is applied to destroy these tumor cells in that an LD/LED field 8, for example having a length of 6 cm long and a width of 1.5 cm, is arranged outside along the large superficial vein 4 downstream, in the direction of blood flow, from the intravascular 405 nm radiation field of the fiber optic cable 10 is activated at an emission wavelength of 630 nm and an output density of 200 mW/cm2 for a treatment period=t1. The high output density 630 nm radiation enters the bloodstream and destroys the tumor cells circulating there.

In the alternative, of course, it is also possible to carry out the PDT using the first fiber optic cable 10 with a substitution from a 405 nm to a 630 nm radiation source and then emitting intravascular 630 nm radiation into the vein 4.

Immediately thereafter it is possible to test the result of the PDT by carrying out the above-described spectral blood analysis (PDD) again.

In the alternative, of course, it is also possible to remove the diagnosed tumor cells in a later treatment step using methods of treatment established in oncology (chemotherapy, radiation therapy).

In the embodiment according to FIG. 4 a device according to the invention comprises an LD/LED field 7 arranged along a vein 4 on the outside of the skin 2 of a patient having, for example a length of 6 cm and a width of 1.5 cm. On the skin 2, downstream from the LD/LED field 7 in the direction of blood flow, the inlet of a fiber optic cable 12 is disposed. The inlet is designed conical so as to enlarge the surface of radiation entry. The outlet of the fiber optic cable 12 is connected to a high sensitivity fluorescence spectrometer or Ramann spectrometer 5 adapted in terms of its spectral position and sensitivity. The spectrometer 5 in turn is connected to a computer 6. A further LD/LED field 8 is arranged downstream from the fiber optic cable 12 in the direction of blood flow, the field being connected to a computer 5 via a line 9. The excitation of a tumor cell enriched with a photosensitizer happens by means of the LD/LED field 7 which emits a corresponding radiation. The fluorescence, phosphorescence, and luminescence radiation generated thereby enters the inlet surface of the fiber optic cable 12 and, from there, is guided to the spectrometer 5.

The diagnostic detection of a tumor cell and its elimination happens in the manner described above. One of the above identified or any other suitable photosensitizer can be utilized as photosensitizer. This is also true for the above described embodiment examples gilt which shall not be restricted to the specifically identified photosensitizers.

1. A device for detecting individual tumor cells, tumor clusters, and micro metastases in the circulatory system, which are enriched with a photo-sensitive substance, the device comprising
a radiation source (7, 11) with intravascular or extravascular excitation of the photo-sensitive substance;
a detector (1, 12, 13) with intravascular or extravascular detection of a fluorescence and/or phosphorescence- and/or luminescence radiation of the excited tumor cells and/or tumor cell clusters and/or micro metastases;
a high sensitivity fluorescence spectrometer (5), connected to said detector (1, 12, 13), for detecting the radiation emitted; and
a computer (6), connected to said spectrometer (5), for recording the received peaks of the radiation emitted as a function of time.

2. The device according to claim 1, wherein said detector (1, 12, 13) is a fiber optic cable.

3. The device according to claim 2, wherein said detector (1, 12, 13) is provided with a conical inlet (14) so as to enlarge the surface for the entry of radiation.

4. The device according to claim 1, further comprising a further radiation source (8, 11) acting intravascularly or extravascularly, arranged, in the direction of blood flow, downstream from said detector (1, 12, 13) and connected to said computer (6), which puts out a switch-on signal to said second radiation source (8, 11) upon detecting a radiation peak.
